(19) United States
(12) Patent Application Publication
(10) Pub. No.: US 2007/0032808 A1
(43) Pub. Date: Feb. 8, 2007

(54) System and method for addressing total occlusion in a vascular environment

(76) Inventors: Azam Anwar, Dallas, TX (US); Paul J. Durfee, Mesquite, TX (US)

Correspondence Address: BAKER BOTTS L.L.P., 2001 ROSS AVENUE, SUITE 600, DALLAS, TX, 75201-2980, US

(21) Appl. No.: 11/197,286

(22) Filed: Aug. 3, 2005

(51) Int. Cl. A61B 17/22

(52) U.S. Cl. 606/159

(57) ABSTRACT

An apparatus for addressing an occlusion in a vascular environment is provided that includes a wire and a tip coupled to the wire and operable to burrow into an occlusion in a vascular environment. The apparatus also includes a coil section disposed between the wire and the tip and operable to collect debris generated by burrowing of the tip.

### TECHNICAL FIELD OF THE INVENTION

This invention relates in general to vascular diseases and, more particularly, to a process, a system, and a method for addressing total occlusion in a vascular environment.

### BACKGROUND OF THE INVENTION

The treatment of vascular diseases has grown exponentially in terms of sophistication and diversity. Procedures involving items such as stents and balloons are virtually routine in most heart-care practices. One problem encountered in many vascular procedures is total occlusion, which is almost impossible to treat, but which remains problematic over the lifetime of any suffering patient.

Stents provide a viable remedy for many problems caused by plaque. In most stent cases, a simple lesion is identified that is inhibiting or restricting blood flow. A balloon is inserted at the targeted location within the patient and then the balloon is inflated. Once the balloon has been properly inflated (potentially several times), the balloon is then removed and a standard stent is conventionally placed. Note that in such an arrangement, the lesion can be easily traversed (i.e. a guide wire can readily cross over the lesion) such that the balloon and stent are positioned at their optimal location with little difficulty.

However, such procedures are impossible in the case of total occlusion, which accounts for a substantial number of cases for most cardiologists. Note that this occlusion issue could be particularly prevalent for previous bypass patients, as native arteries remain clogged. Where a complete impasse exists in the artery, no effective way to treat the blockage currently exists. Hence, stent and balloons are precluded from being used in such an occlusion scenario because the obstruction will not allow the stent to be suitably positioned. Therefore, the ability to properly address total occlusion in a given vascular environment presents a significant challenge for physicians relegated the difficult task of resolving this issue.

### SUMMARY OF THE INVENTION

From the foregoing, it may be appreciated by those skilled in the art that a need has arisen for an improved process for restoring blood flow to areas afflicted by total occlusions. In accordance with an embodiment of the present invention, a process, a system, and a method for addressing an occlusion are provided that substantially eliminate or greatly reduce disadvantages and problems associated with conventional vascular disease approaches, strategies, and instruments.

According to an embodiment of the present invention, an apparatus for addressing an occlusion in a vascular environment is provided that includes a wire and a tip coupled to the wire and operable to burrow into an occlusion in a vascular environment. The apparatus also includes a coil section disposed between the wire and the tip and operable to collect debris generated by burrowing of the tip.

In more particular embodiments, the apparatus includes a drive unit operable to cause rotation of the tip in order to burrow through the occlusion. The apparatus may also include a brake operable to limit movement of the wire. The wire can be advanced by the drive unit in millimeter increments. The apparatus may further include a housing that includes the drive unit and a power source that is operable to supply energy for the drive unit, where at least a portion of the wire is resident in the housing.

Certain embodiments of the present invention may provide a number of technical advantages. For example, according to one embodiment of the present invention, an architecture and a process are provided that allows for an optimal collection of plaque and debris at the arterial site. This is critical because this friable plaque material is detrimental for the patient and, therefore, must be properly accounted for by the attending cardiologist. In particular, the coil section of the system operates as an effective debris gathering tool, whereby particles become embedded in its internal ring structure. This provides a relatively safe solution for handling this dangerous material in the context of addressing total occlusion situations.

The present invention also offers enhanced maneuverability at the targeted occlusion site. This is due, in part, to the inner wire structure of the present invention, which can be manipulated using a wire steering clamp. Moreover, cooperating with fluoroscopy at designated intervals in the procedure, the present invention can yield highly accurate movements of the device. Hence, the system can cooperate with fluoroscopy to ensure safety and to confirm correct placement of the device. Additionally, the present invention is intuitive and easy to operate (i.e. requires minimal training before operating).

The present invention also provides for enhanced versatility in its applications, as it can readily be applied to virtually any occlusion scenario. This could include operations and protocols that involve the carotid artery, the renal artery, the clavicular artery, as well as numerous other vascular regions. Thus, such a device can be used by physicians in cardiac catheterization labs, in special procedure labs, or in other suitable vascular environments in which occlusions are prevalent.

Certain embodiments of the present invention may enjoy some, all, or none of these advantages. Other technical advantages may be readily apparent to one skilled in the art from the following figures, description, and claims.

### BRIEF DESCRIPTION OF THE DRAWINGS

To provide a more complete understanding of the present invention and features and advantages thereof, reference is made to the following description, taken in conjunction with the accompanying figures, wherein like reference numerals represent like parts, in which:

FIG. 1 is a simplified schematic diagram illustrating a system that addresses an occlusion in a vascular environment;

FIG. 2 is a simplified schematic diagram illustrating an example internal structure of the system of FIG. 1;

FIG. 3A is a simplified flowchart illustrating a number of general steps associated with one implementation of the system; and

FIG. 3B is a two-part simplified schematic diagram of an occlusion that is addressed by the system.

### DETAILED DESCRIPTION OF THE INVENTION

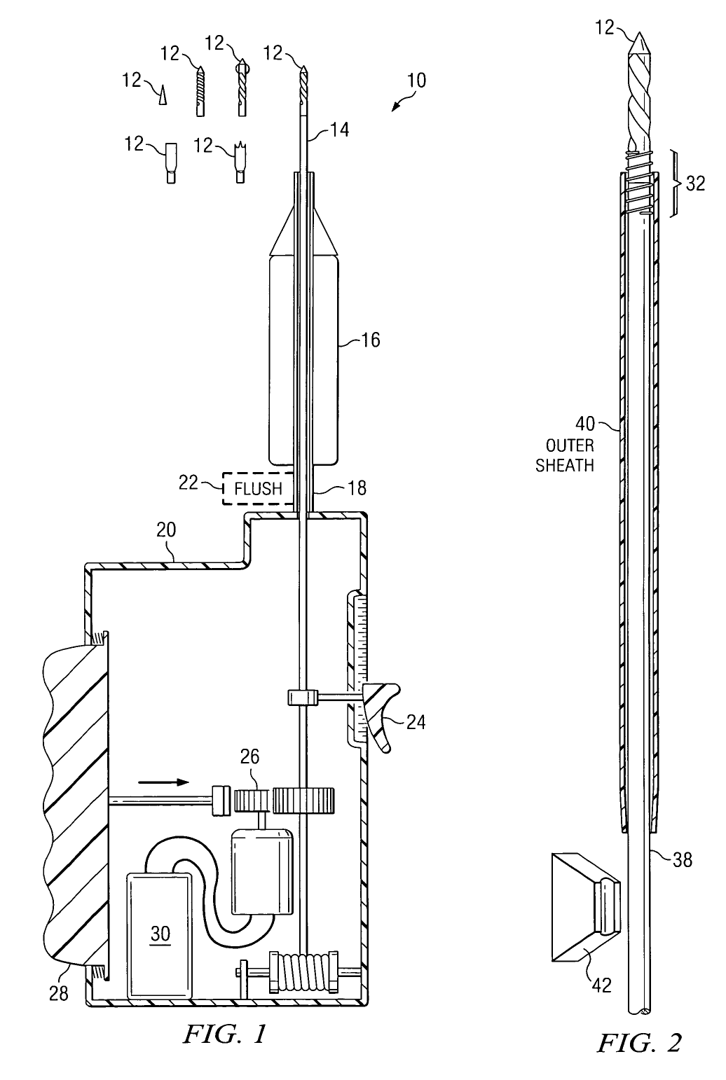

FIG. 1 is a simplified block diagram of a system 10 for treating occlusion in the human body. In one particular application, system 10 can be used to treat chronic total occlusion (CTO) in peripheral and coronary arteries. The peripheral environment represents a somewhat larger environment that is not necessarily susceptible to dangers posed by other areas. In other applications, system 10 can be used for blockages in any area of the body in which an obstruction exists. References to peripheral and coronary arteries in the following examples are only offered for purposes of teaching and, therefore, should not be construed to limit or to restrict the broad uses of the present invention in any way. Other applications could implicate the renal arteries, carotid arteries, clavicular arteries, femoral arteries, etc.

System 10 may include a tip 12, which is attached to a wire 14. Tip 12 may be secured to a clamping element 16 (e.g. a Touey-Borst clamp) that includes a casing 18 in which wire 14 resides. Casing 18 extends from clamping element 16 to a housing 20, which offers a malleable grip for a user of system 10. Included within housing 20 are an advancer 24, a drive unit 26, a brake 28, and a battery 30. Wire 14 may be inserted into housing 20 from the bottom or wire 14 may be spooled within housing 20.

Note that a spring-like reservoir could also be provided to the present invention. This is illustrated as a flush mechanism 22 in FIG. 1. The reservoir would be able to facilitate drilling operations (i.e. provide a lubricant where necessary or provide a flow that could carry debris from the occlusion site). Such a reservoir could be managed by a second attending physician or a triggering mechanism could be provided on the handle of the present invention such that a single individual could operate the device without the assistance of a second party. Hence, flush 22 could simply be part of casing 18, as is illustrated in the example embodiment of FIG. 1.

Tip 12 can be used for purposes of burrowing through a targeted blockage. In one embodiment, tip 12 includes one or more teeth that militates an opening of the blockage, as tip 12 traverses the obstruction. In other embodiments, tip 12 can comprise sharp elements that can cut or incise specific areas in order to achieve a penetration of the occlusion. Considerable flexibility is provided by tip 12, as any number of implementations of tip 12 could produce the desired effect of opening a given conduit.

In order to further elucidate some of the design possibilities of tip 12, a number of example tips are illustrated in FIG. 1. One tip is somewhat sharp, whereas another illustrated in FIG. 1 is more applicable to burrowing (i.e. similar to a mason drill bit design). Other bits could include more concentric drilling ridges, which could be used in finer drilling applications in which progress may be sluggish. Considerable flexibility is contemplated by the design choices of tip 12: all such alternatives being clearly within the broad scope of the present invention.

Tip 12 is both sensing and somewhat intuitive, as it can encounter corners and narrow passageways and navigate those successfully. In one embodiment, tip 12 is tapered such that if an obstacle is placed in front of tip 12, it can readily move around the obstacle (or easily shift directions) before burrowing or perforating in undesired areas. In this sense, tip 12 is integral to the operations of system 10, as further detailed and discussed below. Wire 14 is variable in size (e.g. 0.007 inches to 0.0038 inches in diameter, whereby the standard is generally 0.014-0.018 inches in diameter).

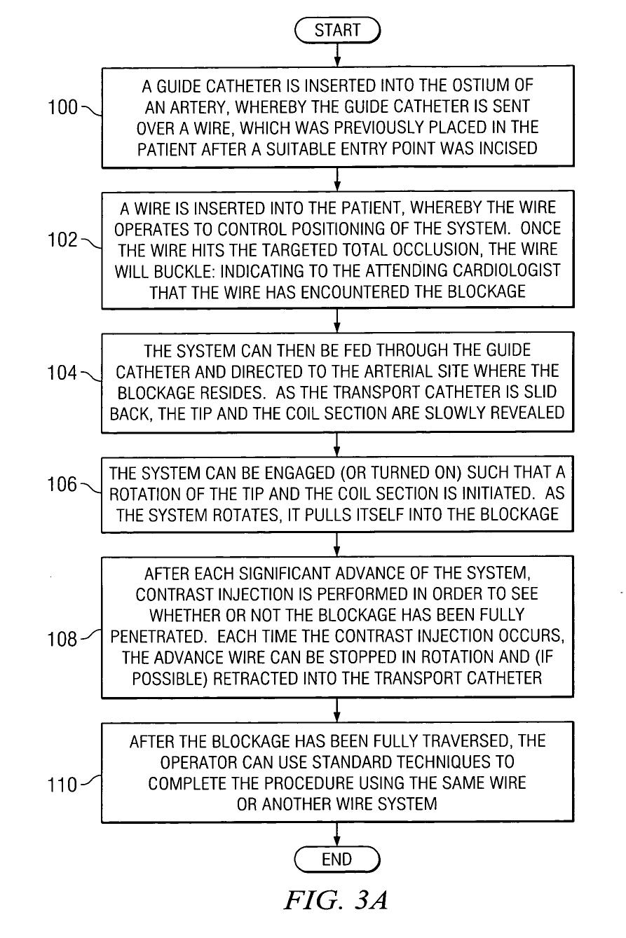

In order to further detail the structure of FIG. 1, reference is now made to FIG. 2, which offers some details associated with the internal architecture of system 10. These two FIGURES should be interpreted together, as they further elucidate the components and the features of the present invention.

Tip 12 can be coupled to a coil section 32 (illustrated in FIG. 2), which can offer an ability for wire 14 to bend and to avoid crimping. This offers the benefit of maneuverability to system 10. Coil section 32 can be exposed in the target area such that it can collect debris (e.g. from plaque), as rotation is produced by system 10. [Note that the rotation speed is variable.] Once the occlusion has been traversed, a prophylactic (e.g. a transport catheter) can be positioned over tip 12 and coil section 32 such that the trapped debris can be safely removed from the patient.

In operation, tip 12 can mimic a drilling pattern, where the shavings are mashed into coil section 32 such that the remnants of the drilling are suitably trapped. Once secured, a simple transport catheter may be used to transfer the debris from the patient.

An inner wire 38 of FIG. 2 (which is the same as wire 14 of FIG. 1) is coupled to tip 12 and is relatively small in comparison to an outer sheath 40, both of which are included in system 10. It should be noted that system 10 could easily be used with standard balloons. For example, outer sheath 40 could be a standard ‘over-the-wire’ balloon.

In one non-limiting embodiment, inner wire 38 may be 14/1000 of an inch in diameter and outer sheath 40 could be 21/1000 of an inch in diameter. Inner wire 38 (also referred to as a “total occlusion wire” (TOW)) can be made of grooved NITINOL or a comparable wire type (braided or non-braided), which can be suitably charged for enhancing the collection activities of the device. Other embodiments can certainly employ other materials for wire construction to achieve the operations of the present invention.

Note that the inner lumen and the outside of the wire should work together freely, whereby a NITINOL wire would facilitate this endeavor. A lubricating material (e.g. Teflon) could also be used in conjunction with these elements. The lubrication system precludes the wire sheath from binding or inhibiting the rotation.

In one example architecture, inner (advancing) wire 38 can be grooved much like a common drill bit. The grooves can serve the purpose of collecting the debris shaved off during operation. Inner wire 38 should also be made in a manner such that tip 12 is directional. This can be accomplished by employing an inner and outer lumen construction of the guide wire. The inner lumen can be attached to the wire head and covered by a spring grooved outer housing. This can offer freedom of motion to the wire tip while engaged. Note that the wire tip can be grooved or non-grooved (i.e. standard): such choices being based on particular circumstances or individual patient needs. In some cases, a smooth dull tipped wire may be utilized (e.g. in the case of a suspect thrombus).

Inner wire 38 can be housed in the introducing or transport catheter, which is a hollow non-balloon tipped catheter that includes a distal radiopaque marker to give a precise location of exit for the advancing wire. The transport catheter can allow an operator to identify where his wire tip is in relation to the most proximal part of the occlusion.

In operation, tip 12 can be positioned at the location where further advancement is desired. Drive unit 26 can then be engaged by placing the distal end of the wire into drive unit 26 and tightening the wire using a simple clamp (which is generally removable). The transport catheter is then advanced and drive unit 26 is subsequently engaged. When inner wire 38 is clamped inside drive unit 26, the advancing knob can be connected to the clamp: allowing the wire to engage the occlusion and to form a channel in the blockage (through rotation). Inner wire 38 can be progressively (and slowly) moved forward, while rotating, via the advancing knob. By engaging inner wire 38, tip 12 can be manipulated left or right: ensuring proper placement without perforation or tissue damage. Debris created by the rotation can be collected in the wire grooves or in coil section 32. Note also that any portion of coil section 32 (or of inner wire 38) may be positively charged to help trap the debris, as these elements successively progress into the occlusion.

A generic wire steering clamp 42 is coupled to inner wire 38. Wire steering clamp 42 can be used to assist in controlling or turning inner wire 38, particularly in cases where, due to blood or other fluids, inner wire 38 becomes difficult to manipulate. Wire steering clamp 42 is important because it effectively alleviates vibrations or turbulence created by system 10 and which normally occurs in the closed system of a patient. Wire steering clamp 42 allows for a somewhat trauma-free procedure, where a certain amount of dexterity and finesse can be employed to obviate sudden unwanted movements. Wire steering clamp 42 is removable.

In operation of one example, drive unit 26 can be actuated by advancer 24, where battery 30 serves as a power source for system 10. In other embodiments, battery 30 may be supplanted by a power cord or any other energy source for system 10. Drive unit 26 may include a set of gears, which can mate with wire 14 (indirectly or directly) in order to precisely advance wire 14 along the chosen pathway. For example, drive unit 26 (in conjunction with advancer 24) could achieve incremental movements in the millimeter range. In order to further guide an end user of system 10, millimeter graduations may be provided along advancer 24, as is illustrated in FIG. 1.

In accordance with one ergonomic design, an end user's thumb can be used to trigger advancer 24, whereby brake 28 can be activated by a collective squeezing of the other fingers of the end user. In other designs, such an arrangement may be inverted such that an index finger is used to trigger advancer 24 and a thumb is used to trigger brake 28.

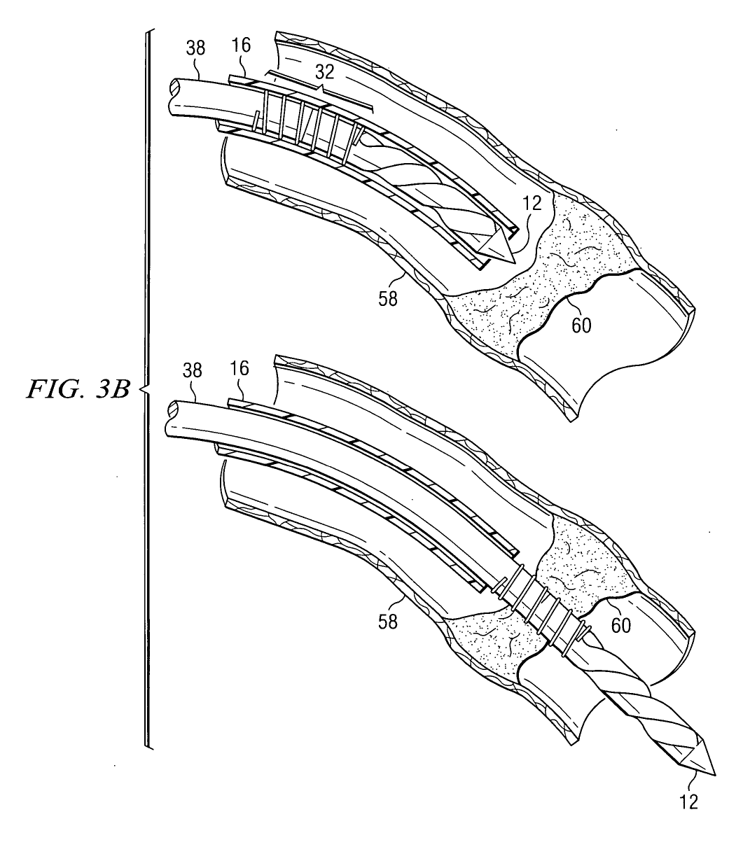

FIGS. 3A and 3B represent related illustrations that should be interpreted together. FIG. 3A is a simplified flowchart that offers one example operation, which could be performed using system 10. FIG. 3B is a simplified block diagram of an occlusion, which is present in the body. FIG. 3B offers a two-part depiction (i.e. a before and an after snapshot) of system 10 being employed. FIG. 3B includes an artery 58, which has a blockage 60 that will be addressed by system 10.

In the example of FIGS. 3A and 3B, the patient has a blockage in his femoral artery. At step 100, a guide catheter is inserted into an ostium of an artery, whereby the guide catheter is sent over a wire, which was previously placed in the patient after a suitable entry point was incised (e.g. in the groin area). The guide catheter can operate as a conduit for items to be positioned at the site. At step 102, a wire is inserted into the patient, whereby the wire operates to control positioning of system 10. Once the wire hits the targeted total occlusion, the wire will buckle: indicating to the attending cardiologist that the wire has encountered the blockage.

The guide catheter (also referred to as an “introducing transport catheter”) is simply a balloon catheter structure, but without the balloon at its end. The shaft is the same, but the tip includes a marker that can be identified through fluoroscopy.

At step 104, system 10 can then be fed through the guide catheter and directed to the arterial site where the blockage resides. As the transport catheter is slid back, tip 12 and coil section 32 are slowly revealed. At step 106, system 10 can be engaged (or turned ON) such that a rotation of tip 12 and of coil section 32 is initiated. As system 10 rotates, it pulls itself into blockage 60. While system 10 pulls itself into blockage 60, the rotation of coil section 32 operates to embed plaque (and any other materials present at the site) into its core and its rings.

After each significant advance of system 10, a contrast injection can be performed in order to see whether or not blockage 60 has been fully penetrated. Short bursts of contrast media will allow the attending physician to know precisely when the occlusion has been bridged. Each time contrast injection is performed, the advance wire can be stopped in rotation and (if possible) retracted into the transport catheter. This will lessen the chance of distal embolization of shaved debris. This is illustrated by step 108.

After blockage 60 has been fully traversed by tip 12 (i.e. the distal part is now open), the guide wire can be positioned through blockage 60 and system 10 may be suitably removed from the patient at step 110. Once blockage 60 has been crossed, the transport catheter can be removed by traditional over-the-wire techniques, leaving the advance wire past the occlusion. Hence, after traversing blockage 60, the operator can use standard techniques to complete the procedure using the same wire or using another wire system.

With its removal, system 10 can take plaque and other debris with it. (Obviously blood thinning agents and other drugs may be employed during the procedure to break down much of this plaque material.) Once system 10 has been safely removed, any number of subsequent procedures may be performed, such as a balloon or stent placement. Thus, once the wire has crossed the occlusion, any further treatment can then be employed to restore blood flow to the area of occlusion, without the necessity of exchanging wires. Note that the transport catheter can easily be grooved on one side to allow for the passage of a second wire if needed.

An ensuing stent procedure would be preferred following the completion of a successful traversal of the occlusion, as such an arterial area would likely experience a subsequent occlusion if not protected. A plain old balloon angioplasty (POBA) would not be recommended without providing the additional protection of a stent. Thus, in a general sense, system 10 could be considered an intermediate step and not necessarily a final-end remedy. System 10 can serve as the proverbial “set-up” for ensuing procedures that address the existing environmental issues associated with the accumulation of plaque.

System 10 may be highly applicable to bypass patients, whose arteries have been re-occluded by plaque. Indeed, previous bypass patients could benefit greatly from such a procedure. This is because system 10 could be used to penetrate native arteries that were previously blocked. Note that the distal portion of a bypass is still viable and, thus, could readily be employed if the blockage is crossed.

Note that a simple kit could be provided to the cardiologist, who is relegated the task of addressing a total occlusion in an artery. The kit could include a drive unit, a TOW wire, a transport catheter, and a suitable steering mechanism. In other embodiments, the kit could include additional elements such as a drug-coated stent, a balloon, etc.

It is important to note that the stages and steps in FIG. 3A illustrate only some of the possible scenarios that may be executed by, or within, the present system. Some of these stages and/or steps may be deleted or removed where appropriate, or these stages and/or steps may be modified or changed considerably without departing from the scope of the present invention. In addition, a number of these operations have been described as being executed concurrently with, or in parallel to, one or more additional operations. However, the timing of these operations may be altered considerably. The preceding example flows have been offered for purposes of teaching and discussion. Substantial flexibility is provided by the tendered architecture in that any suitable arrangements, chronologies, configurations, and timing mechanisms may be provided without departing from the broad scope of the present invention.

Note also that the example embodiments described above can be replaced with a number of potential alternatives where appropriate. The processes and configurations discussed herein only offer some of the numerous potential applications of system 10. The elements and operations listed in FIGS. 1-3B may be achieved with use of system 10 in any number of contexts and applications. Accordingly, suitable infrastructure may be included within system 10 to effectuate the tasks and operations of the elements and activities associated with managing total occlusion.

Although the present invention has been described in detail with reference to particular embodiments in FIGS. 1-3B, it should be understood that various other changes, substitutions, and alterations may be made hereto without departing from the sphere and the scope of the present invention. For example, although the preceding FIGURES have referenced a number of components as participating in the numerous outlined procedures, any suitable equipment or relevant tools may be readily substituted for such elements and, similarly, benefit from the teachings of the present invention. These may be identified on a case-by-case basis, whereby a certain patient may present a health risk factor while another (with the same condition) may not. Hence, tip 12 or coil section 32 may be designed based on particular needs with particular scenarios envisioned. This could include modifying tip 12 by the surgeon ‘in situ’ or prior to performing the procedure.

Numerous other changes, substitutions, variations, alterations, and modifications may be ascertained to one skilled in the art and it is intended that the present invention encompass all such changes, substitutions, variations, alterations, and modifications as falling within the spirit and scope of the appended claims. In order to assist the United States Patent and Trademark Office (USPTO) and additionally any readers of any patent issued on this application in interpreting the claims appended hereto, Applicant wishes to note that the Applicant: (a) does not intend any of the appended claims to invoke paragraph six (6) of 35 U.S.C. section 112 as it exists on the date of filing hereof unless the words “means for” are specifically used in the particular claims; and (b) does not intend by any statement in the specification to limit his invention in any way that is not otherwise reflected in the appended claims.

What is claimed is:

1. An apparatus for addressing an occlusion in a vascular environment, comprising:
a wire;
a tip coupled to the wire and operable to burrow into an occlusion in a vascular environment; and
a coil section disposed between the wire and the tip and operable to collect debris generated by burrowing of the tip.

2. The apparatus of claim 1, further comprising:
a drive unit operable to cause rotation of the tip in order to burrow through the occlusion.

3. The apparatus of claim 2, further comprising:
a brake operable to limit movement of the wire.

4. The apparatus of claim 2, wherein the wire can be advanced by the drive unit in millimeter increments.

5. The apparatus of claim 2, further comprising:
a housing that includes the drive unit and a power source that is operable to supply energy for the drive unit, wherein at least a portion of the wire is resident in the housing.

6. The apparatus of claim 1, wherein the housing allows for the apparatus to be hand held such that actuation and braking of the drive unit can be achieved by an end user employing a single hand.

7. The apparatus of claim 1, further comprising:
a wire steering clamp coupled to the wire and operable to control a direction of the wire.

8. The apparatus of claim 1, wherein the wire comprises NITINOL.

9. The apparatus of claim 1, wherein the tip is tapered and the tip includes edges that facilitate a drilling of the occlusion.

10. The apparatus of claim 1, further comprising:
a transport catheter operable to carry the wire, the coil section, and the tip away from the occlusion in order to assist in removing the debris.

11. A method for addressing an occlusion in a vascular environment, comprising:
positioning a tip at a total occlusion site, wherein the tip is coupled to a wire and is operable to burrow into an occlusion at the site;
providing a rotation for the tip such that burrowing is achieved; and
collecting debris generated by burrowing of the tip.

12. The method of claim 11, further comprising:
activating a drive unit operable to cause rotation of the tip in order to burrow through the occlusion.

13. The method of claim 11, further comprising:
limiting movement of the wire through a braking mechanism.

14. The method of claim 11, further comprising:
advancing the wire in millimeter increments.

15. The method of claim 11, further comprising:
performing contrast injection at the occlusion site at intermittent periods in order to confirm placement of the tip.

16. The method of claim 11, further comprising:
positioning a transport catheter over the wire, the tip, and a coil section disposed therebetween before exiting the occlusion site.

17. The method of claim 16, further comprising:
placing a stent at the occlusion site after the occlusion has been traversed by the tip.

18. The method of claim 16, further comprising:
utilizing a balloon at the occlusion site after the occlusion has been traversed by the tip.

19. The method of claim 11, further comprising:
loading the wire in a spool configuration included in a housing for a system associated with the wire and the tip.

20. The method of claim 11, further comprising:
manipulating the wire and the tip using a handheld device that provides for advancement and braking of the wire.
